(19) United States
(12) Patent Application Publication
(10) Pub. No.: US 2017/0051050 A1
(43) Pub. Date: Feb. 23, 2017

(54) POLYMERIC SUBSTRATES WITH IMMOBILIZED ANTIBODIES AND METHOD OF PRODUCTION THEREOF

(71) Applicant: ASSOCIATION FOR THE ADVANCEMENT OF TISSUE ENGINEERING AND CELL BASED TECHNOLOGY AND THERAPIE, BRAGE (PT)

(72) Inventors: Albino Manuel PEREIRA MARTINS, ANTIME (PT); Ana Catarina FREITAS SALAZAR DE OLIVEIRA, SAO MARTINHO SANDE (PT); Rui Luís GONÇALVES DOS REIS, PORTO (PT); Nuno João MELEIRO ALVES DAS NEVES, BRAGA (PT)

(21) Appl. No.: 15/307,698

(22) PCT Filed: Apr. 28, 2015
(86) PCT No.: PCT/IB2015/053091

§ 371 Date: Oct. 28, 2016

(30) Foreign Application Priority Data
Apr. 28, 2014 (PT) 107609

(51) Int. Cl. C07K 16/22; C07K 17/10; A61K 47/48; G01N 33/545; G01N 33/548; C07K 17/08; G01N 33/543

(52) U.S. Cl. CPC C07K 16/22; C07K 17/08; C07K 17/10; G01N 33/54353; G01N 33/545; G01N 33/548; A61K 47/48984; A61K 47/4823; A61K 47/482

(57) ABSTRACT

The present invention relates to biofunctional polymeric substrates product comprising immobilized antibodies that selective bind to antigens namely autologous growth factors and an inflammatory molecule, uses and method of production. The bio-functional polymeric substrate can be used in a quantification method of antigens namely soluble growth factors, inflammatory molecules and hormones or the biofunctional polymeric substrate can be used to isolate extracellular vesicules. The biofunctional polymeric substrate can be also used in Tissue Engineering and Regenerative Medicine approaches, namely in bone and cartilage lesions, esophageal lesions, periodontal ligament reconstruction and skin lesions regeneration or treatment.

### TECHNICAL FIELD

The present invention relates to biofunctional polymeric substrates product comprising immobilized antibodies that selective bind to antigens namely autologous growth factors and an inflammatory molecule, uses and method of production.

The biofunctional polymeric substrate can be used in a quantification method of antigens namely soluble growth factors, inflammatory molecules and hormones.

In another embodiment, the biofunctional polymeric substrate can be used to isolate extracellular vesicles, in particular microvesicles.

The biofunctional polymeric substrate can be also used in Tissue Engineering and Regenerative Medicine approaches, namely in bone and cartilage lesions, esophageal lesions, periodontal ligament reconstruction and skin lesions regeneration or treatment.

### BACKGROUND ART

The biofunctionalization of surfaces gained special interest by the need to optimize the biological integration of implantable medical devices. Protein immobilization requires that the substrate should provide a biocompatible and bioactive environment, since it should interact positively with the native structure of the proteins and biomolecules. Of particular interest is the immobilization of antibodies, due to the high specific interaction with their ligand molecules, making a more efficient and irreversible attachment of the target protein and/or ligand.

Different immobilization methods and strategies can be implemented to achieve the biofunctionalization of substrates such as physical adsorption, covalent immobilization or antibody-binding proteins. Covalent immobilization is the most reported method, since it leads to a strong and stable attachment. In this method the presence of functional groups is required in both the substrate and the molecule to be immobilized. Due to the use of coupling agents, modifications of the antigen binding site can occur which may cause loss of functionality of the antibodies.

Proteins, especially growth factors (GFs), have an important role in the regulation of a variety of cellular processes, as well as on the coordination of the healing process of different tissues. GFs functions and purposes range from inducing vascularization and angiogenesis to cell growth, proliferation and differentiation. Therefore, the local or systemic administration of GFs may be a valuable therapeutic approach in the successful treatment of different chronic wounds. Clinically, GF-based strategies are applied in plastic surgery, bone and cartilage lesions, muscle injuries, ulcers and skin regeneration. Particularly, biological fluids such as the Platelet Lysate (PL), that consist of a cocktail of different GFs, e.g. basic fibroblast growth factor (bFGF), vascular endothelial growth factor (VEGF), transforming growth factor beta 1 (TGF-β1), platelet-derived growth factor subunit B (PDGF-BB), epidermal growth factor (EGF), insulin-like growth factor 1 (IGF-I), provide a complex mixture of chemical signals to the cells at the injury site, which is often associated with a non-specific action.

### General Description

The present invention relates to a biofunctional polymeric substrate comprising immobilized antibodies that selectively bind to specific antigens, in particular growth factors—GFs, inflammatory molecules, hormones, extracellular vesicles, among others, specific uses and method of production thereof, thus taking advantage of the specific and efficient interactions between a specific antibody and its antigen. With this solution it is possible, for example, to selectively immobilize from a pool of highly concentrated GFs present in the PLs—Platelet Lysate, just the ones of interest for the envisioned application, i.e. bFGF, TGF-β1 and VEGF. Herewith, an antigen can thus be defined as a substance that can combine specifically with an antibody.

Furthermore, this biofunctionalization strategy also enables the simultaneous immobilization of multiples antibodies at a time, distributed in a mixed or in a side-by-side fashion over the same polymeric substrate, in particular fibrous, enabling designing studies to elucidate the synergistic effect of the combined GFs.

The immobilization of biomolecules at the surface of different biomedical devices has attracted enormous interest, in order to enhance their biological functionality at the cellular level, this is an in situ funcionalization.

Therefore, as extracted from the state of the art exposed above, it would be desirable to have a single substrate that is able to immobilized antibodies of different parts of the patient body, in this case is no need to have an specific substrate (mesh, membrane) for each specific place of the body.

The present disclosure refers to a biofunctional polymeric substrate, in particular a fibrous polymeric substrate product, comprising a polymeric substrate capable of selectively bind Growth factors (GFs) of interest from a pool of proteins present in a biological fluid, such as a Platelet Lysate (PL). The present disclosure further refers to the method of production of this biofunctional polymeric substrate product.

The present disclosure describes a polymeric substrate product/composition for binding to antigens, wherein said product comprises a polymeric substrate having a specific surface area between 15-90 cm²/mg, said substrate comprising:
a functional group able to bind to an antibody, wherein the functional group is selected from a list consisting of: amine, sulfhydryl, carbonyl, carboxyl, or mixtures thereof, and
an antibody for binding to an antigen, preferably a plurality of antibodies,
   wherein the antibody is bound to the substrate through said functional group and the antibody concentration is more than 1 μg/mL, in antibody mass per biologic fluid volume.

The polymeric substrate described in the present disclosure is capable of selectively binding to a specified antigen and thus provide the selected bound antigen in tissue treatment and regeneration body locations. This way, naturally occurring substances in the body fluids, particularly blood plasma, synovial fluid, saliva, urine, blood serum, cerebrospinal fluid (CSF), pericardial fluid, peritoneal fluid, pleural fluid, amniotic fluid, breast milk, seminal fluid, vaginal secretion, lymph, endolymph, perilymph, sebum, cerumen, mucus, exudates, bile, tears, sweat, aqueous and vitreous humor, and cytosol, can be targeted by the substrate antibody and thus maintained for longer periods of time or in higher concentrations in the target locations an amplification effect of said substances.

Furthermore, when a plurality of antibodies is used, a plurality of antigens can be targeted. For example, a plurality of GFs, hormones, inflammatory agents, etc can be bound. This way, for a specific tissue or for a specific body location, a plurality of substances can be bound and maintained by the substrate, wherein at least one of the substances is compatible with the specific tissue or location treatment or regeneration. Thus, the same substrate can be used in different tissues, without the need to develop specific substrates for each different use.

In a synergic way the covalent immobilization of the antibody to the substrate does not compromise the bioavailability of the antigen binding site and therefor enables the binding of the antigen during the whole treatment.

The antibody concentration is in μg/mL and represents the antibody mass per biologic fluid volume. The antibody concentration, namely can be measured by standard methods, namely by an indirect method consisting in the fluorescence quantification of unbound secondary antibody solution.

The specify area can be measured by standard methods, namely by the gas permeability method, e.g. by the Brunauer-Emmet-Teller method, or mathematically approximated by complex models, e.g. from Eichhorn & Sampson.

In an embodiment of polymeric substrate product disclosed the antibody concentration is more than 1.5 μg/mL, in particular 1.5 μg/mL-20 μg/mL in antibody mass per biologic fluid volume.

In an embodiment of polymeric substrate product disclosed the antibody concentration may be 2 μg/mL-12 μg/mL, preferably 4-8 μg/mL, in particular 3 μg/mL, 4 μg/mL, 5 μg/mL, 6 μg/mL, 7 μg/mL, 8 μg/mL, 9 μg/mL, 10 μg/mL, 2 μg/mL in antibody mass per biologic fluid volume.

In an embodiment of polymeric substrate product disclosed the specific surface area could vary between 20-90 cm²/mg, preferably 40-90 cm²/mg, more preferably 50-90 cm²/mg, even more preferably 60-90 cm²/mg.

In an embodiment of polymeric substrate product disclosed the synthetic polymer may be selected from a list consisting of: polycaprolacton (PCL), polyhydroxyalkanoate (PHA), polyhydroxybutyrate (PHB), polyethylene adipate (PEA), polybutylene succinate (PBS), poly(3-hydroxybutyrate-co-3-hydroxyvalerate)(PHBV), polyethylene terephthalate (PET), poly(ethylene) (PE), poly(styrene) (PS), poly(tetrafluoroethylene) (PTFE), poly(propylene) (PP), poly(pyrrole) (PPY), poly(dimethyl siloxane) (PDMS), poly(methyl methacrylate) (PMMA), polydioxanone (PDA), or mixtures thereof, among others.

In an embodiment of polymeric substrate product disclosed, the polymeric substrate may further comprising a natural polymer selected from starch, chondroitin sulfate, hyaluronic acid, chitosan, alginate, dextran, agarose, cellulose, collagen, xanthan, carrageenan, aloe vera, gelatin, silk protein, fibronectin, or mixtures thereof.

In an embodiment of polymeric substrate product disclosed, the polymeric substrate may comprises the following mixtures of a synthetic and a natural polymer: polycaprolactone and starch, or polycaprolactone and hyaluronic acid, or polycaprolactone and chondroitin sulfate, or polycaprolactone and chitosan, or polycaprolactone and collagen, or polycaprolactone and gelatin, or polycaprolactone and silk protein, or polycaprolactone and fibronectin.

In an embodiment of polymeric substrate product disclosed the antigen may be an autologous growth factor or inflammatory molecule, or extracellular vesicle in particular a microvesicle, or an hormone from a biological fluid.

In an embodiment of polymeric substrate product disclosed, the antibody may be bound by physical adsorption, covalent immobilization or antibody-binding proteins, preferentially covalently bound to the fibrous substrate surface.

In an embodiment of polymeric substrate product disclosed, the surface of the substrate may be activated to introduce a functional group by different routine techniques namely: UV irradiation, plasma, corona discharge, photons, electron beam, ion beam, X-ray, gams-ray, or wet chemistry, among others.

In an embodiment of polymeric substrate product disclosed, for a better bound of the antibody the covalent bound can be mediated by a coupling agent. The coupling agent may be selected from the list: EDC, NHS, Sulfo-NHS, EDC-HCl (hydrogen chloride), sulfo-SANPAH-Succinimidyl ester-phenyl azide, sulfo-LC-SPDP (sulfosuccinimidyl 6-(3′-[2-pyridyldithio]-propionamido)hexanoate), cyanogen bromide (CNBr), sodium cyanoborohydride (NaCNBH3), carbonyl diimidazole (CDI) or mixtures thereof, among others.

In an embodiment of polymeric substrate product disclosed, could comprise at least one antibody against a growth factors may be selected from the list: TGF-α, TGF-β1, TGF-β2, TGF-β3, EGF, HIE, PDGF-AA, PDGF-AB, PDGF-BB, PDGF-CC, PDGF-DD, VEGF-A, VEGF-B, VEGF-C, VEGF-D, IGF-1, FGF-2, FGF-18, BMP-2, BMP-4, BMP-6, BMP-7/OP-1, CDMP-1/GDF-5, CDMP-2, or mixtures thereof.

In an embodiment of polymeric substrate product disclosed, could comprise at least one antibody against an inflammatory molecule selected from the list: tumor necrosis factor-alpha (TNFalpha), granulocyte-macrophage colony-stimulating factor (GM-CSF), interferon-gamma (IFN-gamma), IL-1, IL-6, IL-7, IL-8, IL-10, IL-12, IL-15, IL-17A, IL-17F, IL-18, IL-19, IL-20, IL-21, IL-22 IL-23, IL-32, IL-33 (interleukins) or mixtures thereof.

In an embodiment of polymeric substrate product disclosed could comprise at least one antibody against an hormones selected from the list: prolactin, adrenocorticotropic hormone, angiotensinogen, thyroid-stimulating hormone, parathyroid hormone, Luteinizing hormone, follicle-stimulating hormone, melanocyte-stimulating hormone, thymosin, orexin, leptin, adiponectin, secretin, histamin, endothelin, gastrin, ghrelin, erythropoietin, oxytocin, vasopressin (antidiuretic hormone), proopiomelanocortin, triiodothyronine, thyroxine, calcitonin, parathormone, cholecystokinin, thyrotrophic hormone, growth hormone, corticotrophin, gonadotropin, melatonin, serotonin, epinephrine (adrenaline), norepinephrine (noradrenaline), dopamine, dehydroepiand-rostendione, estrogen, progesterone, testosterone, dihydrotestosterone, estradiol, insulin, glucagon, cortisol, cortisone, corticosterone, aldosterone, inhibin or mixtures thereof.

In an embodiment of polymeric substrate product disclosed could comprise at least one antibody against a surface marker of an extracellular vesicle selected from the list: CD2-associated protein (CD2AP), CD9 molecule, CD14 molecule, CD24 molecule, CD24a antigen (Cd24a), CD36 molecule (thrombospondin receptor), CD44 molecule (Indian blood group), CD55 molecule (decay accelerating factor for complement) (Cromer blood group), CD59 molecule (complement regulatory protein), CD63 molecule, CD81 molecule, CD82 molecule, CD86 molecule, CD274 molecule, CD5 molecule-like (CD5L), CD163 molecule-like 1 (CD163L1), aquaporin 2 (collecting duct) (AQP2), flotillin 1 (FLOT1), Fas ligand (TNF superfamily, member 6) (FASLG), lysosomal-associated membrane protein 1 (LAMP1), lysosomal-associated membrane protein 2 (LAMP2), mannosyl-oligosaccharide glucosidase (MOGS), flotillin 1 (FLOT1), flotillin 2 (FLOT2), intercellular adhesion molecule 1 (ICAM1), tumor susceptibility gene 101 (TSG101), transferrin receptor (p90, CD71) (TFRC) or mixtures thereof.

In an embodiment of polymeric substrate product disclosed, the polymeric substrate could comprise e mixture of two or more antibodies from each group of antibodies against a growth factor, or inflammatory molecule, or extracellular vesicle, or hormone present in a biological fluid: TGF-β1 and TNFalpha, or BMP-2 and estrogen, or FGF-2 and CD63, or IL-1 and cortisol, or TNFalpha and CD9, or estrogen and CD81, or TGF-β1 and TNFalpha and estrogen, or TNFalpha and estrogen and CD9, or TGF-β1 and IL-1 and CD63, or BMP-2 and estrogen and CD81. More in particular the polymeric substrate could comprise e mixture of two or more autologous antibodies from the previous list.

In an embodiment of polymeric substrate product disclosed, the antibodies of the polymeric substrate can be spatially distributed in a random or side-by-side fashion.

In an embodiment of polymeric substrate product disclosed, the polymeric substrate can be fibrous.

In another embodiment of polymeric substrate disclosed, the polymeric substrate can be used in veterinary and human medicine, namely for use in the treatment of diseases that involve the repair or treatment of tissues wherein the tissue to be treated or repair can be skin, cartilage, periodontal ligament, or esophageal submucosa.

In another embodiment of polymeric substrate disclosed, the polymeric substrate can be use as a cell culture polymeric substrate, including co-culture systems.

In another embodiment of polymeric substrate disclosed can be a strip, a net fibers bundle, a mesh or a membrane. The mesh, may be woven or nonwoven.

Other embodiment disclosed the skin patch comprising the polymeric substrate described in the present disclosure.

Other embodiment disclosed a diagnostic kit comprising the polymeric substrate described in the present disclosure.

Other aspect of the present disclosure is a polymeric substrate composition for binding to antigens for use in the regeneration or treatment of tissues, wherein said composition comprises:
a polymeric substrate having a functional group able to bind to an antibody, wherein the functional group is selected from a list consisting of: amine, sulfhydryl, carbonyl, carboxyl, or mixtures thereof, and
an antibody for binding to an antigen,
wherein the antibody is bound to the substrate through said functional group and the antibody concentration is more than 1 μg/mL, in antibody mass per biologic fluid volume.

In other embodiment, different antibodies are previously immobilized at the substrate surfaces, taking advantage of the specific binding between an antibody and its correspondent antigen. In an embodiment, to achieve that purpose, the surface of electrospun polycaprolactone (PCL) fibers is activated and functionalized in order to insert chemical groups for the immobilization of antibodies.

In another embodiment, after determining the maximum immobilization capacity of each antibody, for example is 12 μg/mL for TGF-β1, 8 μg/mL for bFGF and 4 μg/mL for VEGF, the next step is to bind the correspondent GF. Using recombinant proteins almost 100% of the initial concentration is immobilized, whereas for PL-derived GFs the efficiency is of 84-87% for TGF-β1, 55-64% for bFGF and 50-59% for VEGF.

In another embodiment, with this immobilization method it is possible used the substrate in the quantitative measurement of either natural or recombinant proteins in a wide and higher efficiency than the ones already available. Cellular assays in an embodiment confirm the biological activity of the bound VEGF, both recombinant and PL-derived. Multiple antibodies, i.e. bFGF and VEGF, can also be immobilized over the same structure in a mixed or side-by-side fashion. Using both biological fluids and cells from autologous sources, and using this platform, it is possible to implement very effective and personalized therapies, tailored for the needs of specific patient conditions.

Throughout the description and claims the word “comprise” and variations of the word, are not intended to exclude other technical features, additives, components, or steps. Additional objects, advantages and features of the invention will become apparent to those skilled in the art upon examination of the description or may be learned by practice of the invention. The following examples and drawings are provided by way of illustration, and they are not intended to be limiting of the present invention. Furthermore, the present invention covers all possible combinations of particular and preferred embodiments described herein.

### BRIEF DESCRIPTION OF THE DRAWINGS

The following figures show embodiments for illustrating the invention showing:

FIG. 1: a schematic representation of a compartmental watertight device.

FIG. 2 diagrams of the EDC/NHS ratio and concentrations optimization, wherein a) shows the optimization of the coupling agents 1-Ethyl-3-(3-dimethylaminopropyl)carbodiimide (EDC) and hydroxysuccinimide (NHS) ratio; b) shows the optimization of the individual EDC and NHS concentrations, maintaining the previously optimized ratio 1:4; and c) shows the optimization of the final concentration of the EDC/NHS mixture, i.e 50 mM EDC+200 mM NHS, optimized before, in the antibody solution.

FIG. 3 diagrams of the maximum immobilization capacity of a single antibody at the surface of activated and functionalized electrospun nanofiber, wherein a) shows the immobilization of anti-Transforming Growth Factor (TGF)-β1; b) shows the immobilization of anti-basic Fibroblast Growth Factor (bFGF); and c) shows the immobilization of anti-Vascular Endothelial Growth Factor (VEGF).

FIG. 4 comparative diagram of the maximum immobilization capacity of anti-VEGF at the surface of electrospun PCL chitosan nanofibers.

FIG. 5 diagrams of standard curves for single antibody immobilization at the surface of activated and functionalized electrospun nanofibers, wherein a) shows a TGF-β1 antibody standard curve varying between 0 μg/mL and the maximum concentration that can be immobilized, i.e. 12 μg/ml; b) shows bFGF antibody standard curve ranges from 0 μg/mL to 8 μg/mL; and c) shows a VEGF antibody standard curve that varies between 0 μg/mL and 4 μg/mL.

FIG. 6 images of spatial distribution of immobilized primary antibodies at the surface of activated and functionalized electrospun nanofibers.

FIG. 7 diagrams of capability of the biofunctionalized nanofibrous substrate to bind different concentrations of the recombinant protein, wherein a) refers to TGF-β1, b) refers to bFGF, and c) refers to VEGF.

FIG. 8—Optimization of antibody immobilization against an inflammatory molecule. (A) negative control. (B) Maximum immobilization capacity of the anti-TNF-α at the surface of activated and functionalized electrospun nanofibers. (C; D) Spatial distribution of immobilized anti-TNF-α, at a concentration of 6 μg/ml, at the surface of activated and functionalized electrospun nanofibers.

FIG. 9—Concentration of Human Recombinant TNF-α present in the conditioned medium of activated human macrophages, measured by ELISA.

FIG. 10 diagrams of biochemical performance of the endothelial cell line cultured on unmodified electrospun polycaprolactone (PCL) nanofiber mesh (NFM), NFM with immobilized VEGF antibody (NFM_Ab1), both in endothelial cells growth supplemented (ECGS) medium; NFM with immobilized VEGF antibody (NFM_Ab2), NFM with bound recombinant VEGF (NFM+VEGF_{Rec}), Platelet Lysate-derived VEGF (NFM+VEGF_{PL}) with non supplemented media, wherein a) refers to cell proliferation, b) refers to cell viability, c) refers to total protein synthesis, and d) refers to intracellular VEGF synthesis.

FIG. 11 diagrams wherein a) shows quantification of mixed immobilized bFGF and VEGF antibodies, b) shows relative quantification of bound GFs, i.e. VEGF and bFGF, derived from PL.

FIG. 12—images of spatial distribution of the mixed immobilized primary antibodies at the surface of a single activated and functionalized nanofibrous substrate.

FIG. 13—an image of a laser scanning confocal microscopy image demonstrating the side-by-side antibodies immobilization over the same activated and functionalized nanofibrous substrate.

### DETAILED DESCRIPTION

The specific surface area of the polymeric substrate product of the present subject matter can be measured by standard methods, namely by the gas permeability method, e.g. by the Brunauer-Emmet-Teller method, or mathematically approximated by complex models, e.g. from Eichhorn & Sampson.

For this work, an own simplified estimation model of specific calculated surface area (CSA) of fibers mesh was derived as follows. This method has identified limitations in its application: (i) it neglects the presence of any inter fiber connections (covered surface points), (ii) rough fiber surfaces or any fiber diameter distribution. The model needs the input of the fibrous scaffold mass (m_{substrate}) the average fiber diameter (to) and the polymer density (ρ_{PCL}).

By knowing the fibrous substrate mass and the polymer density, the apparent raw material volume of a polymeric porous substrate can be determined by the following equation:

$${V}_{PCL} = \frac{{m}_{Substrate}}{{ρ}_{PCL}}$$

Based on the assumption that the raw material volume shall fit into single cylindrical-shaped fibers with specific length, the volume of a geometrical cylinder is presented by the equation:

$${V}_{Cylindric} = π \cdot l \cdot {( \frac{ω}{2} )}^{2}$$

The two previous equations can be simplified, yielding to the length of a single fiber:

$$l = \frac{4}{π \cdot {ω}^{2}} \cdot \frac{{m}_{Substrate}}{{ρ}_{PCL}}$$

Assuming that the calculated surface area is represented by the lateral surface of a cylindrical-shaped fiber:

$$CSA = 2 π \cdot l \cdot ( \frac{ω}{2} )$$

The equation to determine the calculated surface area can be obtained:

$$CSA = \frac{4}{{ρ}_{PCL}} \cdot \frac{{m}_{Substrate}}{ω}$$

Thus, keeping the mass of the scaffold at a constant values, the specific surface area of mass is given respectively as:

$${S}_{m} = \frac{4}{{ρ}_{PCL}} \cdot \frac{1}{ω}$$

The average fiber diameter (ω) was calculated by the measurement of fibers diameter in five independent images of scanning electron microscopy at 10000×, through the software AxioVision LE v4.8.2.0.

In an embodiment of the polymeric substrate product of the present subject matter the materials may comprise: Polycaprolactone (PCL; Mw=70 000-90 000 determined by GPC), Chloroform, N,N-Dimethylformamide (DMF), hexamethylenediamine (HMD), 1-Ethyl-3-(3-dimethylaminopropyl)carbodiimide (EDC) and hydroxysuccinimide (NHS) from Sigma Aldrich and use as received. Mouse anti-Human TGF-β1 monoclonal antibody from PrepoTech Inc. (Rochy Hill, N.J.; USA), rabbit anti-Human bFGF oligoclonal antibody (clone 7HCLC), ABfinity recombinant, from Life Technologies (Carlsbad, Calif.; USA); and mouse-anti-Human VEGF (JH121) from Santa Cruz Biotechnology Inc. (Santa Cruz, USA). Regarding the secondary antibodies, both Alexa Fluor® 488 donkey anti-rabbit IgG (H+L) and Alexa Fluor® 594 goat anti-mouse IgG (H+L) are from Life Technologies (Carlsbad, Calif.; USA). The growth factors (GFs), namely the recombinant human TGF-β1, recombinant basic-FGF and recombinant human VEGFin are all from PrepoTech Inc. (Rochy Hill, N.J.; USA).

### Electrospinning of Polycaprolactone Nanofiber Meshes

In an embodiment of the polymeric substrate product of the present subject matter the nanofiber meshes can be obtain by electrospinning, activation and functionalization as following describe.

In an embodiment, a 17% (w/v) PCL solution is prepared with an organic solvent mixture of Chloroform and DMF in a 7:3 ratio as described in Martins, A. et al. Surface modification of electrospun polycaprolactone nanofiber meshes by plasma treatment to enhance biological performance. Small 5, 1195-206 (2009). The PCL solution is electrospun by applying a voltage of 13.6 kV, a needle tip to ground collector distance of 18 cm and a flow rate of 1 mL/h. After the complete processing of 1 mL of PCL solution, the nanofiber mesh (NFM) is allowed to dry for 1 day. This processed NFM is cut into samples of 1 cm×1 cm for further assays.

### Ultraviolet-Ozone Irradiation and Aminolysis

In an embodiment, for the activation of the nanofibers surface, an ultraviolet-Ozone (UV-Ozone) cleaner system is used (ProCleaner™ 220, Bioforce Nanoscience). Both sides of the electrospun NFMs are exposed during 4 minutes to UV-Ozone irradiation, as optimized previously. After this surface activation, amine groups (—NH₂) are inserted at the surface of electrospun nanofibers by immersion in a 1 M HMD (Sigma Aldrich) solution during 1 h at 37° C. The amount of NH₂ (2.83±0.11 nmol/cm²) is determined indirectly by quantifying the amount of free SH groups according to Ellman's reagent method ° as described in Monteiro N., Martins A., Pires R. A., Faria S., Fonseca N. A., Moreira J. N., Reis R. L., and N. N. M. Immobilization of bioactive factor-loaded liposomes at the surface of electrospun nanofibers targeting tissue engineering strategies. (2013).

### Electrospinning of Chitosan Nanofiber Meshes

In an embodiment of the polymeric substrate product of the present subject matter the nanofiber meshes can be obtain by the electrospinning of chitosan nanofiber meshes (Ch NFM) was successfully achieved by the use of a 6 wt. % chitosan solution. Briefly, the powder purified chitosan was added to a solvents mixture of Trifluoroacetic acid (TEA; Sigma) and Dichloromethane (DCM; Sigma), in a volume ratio of 7:3, respectively. Before electrospinning, the solution was left under stirring overnight at room temperature. The processing of that polymeric solution was carried out using a flat vegetal foil as ground collector. The capillary tip-to collector distance and the flow rate were fixed in 12 cm and 0.8 ml/h, respectively. The applied voltage was in the range of 20-25 kV. Neutralization of the electrospun chitosan nanofibers meshes was carried out by immersing the meshes in Ammonia 7N (Aldrich) aqueous solution for 10 minutes at room conditions. After the immersion, the meshes were repeatedly washed with distilled water until neutral pH was obtained. After washing, they were frozen at −180° C. and lyophilized. The Ch NFMs were cut in samples with areas of approximately of 1 cm² and sterilized by ethylene oxide. For chitosan there is no need for chemical treatment, once, it already has NH₂ groups on its chemical structure.

### Antibodies Immobilization

In an embodiment of the polymeric substrate product of the present subject matter the EDC/NHS ratio and concentrations can be optimized.

In an embodiment, EDC/NHS reagents are dissolved in 0.1 MES (2-(N-morpholino) ethanesulfonic acid) buffer with 0.9% (wt/wt) NaCl, following pH adjustment to 4.7, and mixed for 15 min at RT for antibody activation. Five different EDC/NHS ratios are tested, i.e. 1:4, 1:2, 1:1, 2:1 and 4:1, and the optimized ratio is further assayed at four different concentrations (10 mM EDC+40 mM NHS, 26 mM EDC+104 mM NHS, 50 mM EDC+100 mM NHS and 100 mM EDC+400 mM NHS). With the optimized reaction conditions, in terms of EDC/NHS ratio and respective concentrations, the final concentration of the linker in the antibody solution is determined for 1%, 5% and 10% concentrations.

Optimization of Single Immobilization and Determination of the Standard Curves

In an embodiment, three different antibodies are immobilized (anti-TGF-β1, anti-bFGF and anti-VEGF) at the surface of the activated and functionalized electrospun nanofiber meshes. A wide range of primary antibody concentrations, from 0 μg/mL to 20 μg/mL, is tested to find out the maximum immobilization capacity of the nanofibrous substrate. The electrospun NFMs are incubated with 200 μl of each primary antibody concentration. After overnight incubation at 4° C., each mesh is washed twice with 300 μl 0.1 M PBS (5 min each time) and a blockage of 3% BSA is performed for 30 minutes at RT. The BSA solution is removed and the secondary antibody (1:200 in PBS) incubated for 1 h at RT. In order to determine the degree of immobilization, an indirect method is used to quantify the fluorescence of unbound secondary antibody solution (n=3 samples, read in triplicate). For the TGF-β1 and VEGF antibodies, Alexa Fluor® 594 is used and the reading parameters are the absorption at 590 nm and the emission at 617 nm. In the case of the anti-bFGF, the selected secondary antibody is the Alexa Fluor® 488 and the reading parameters are 495 nm for the adsorption and 519 nm for the emission spectrum. Negative control samples are also prepared, where all antibody immobilization steps are performed with the exception of the primary antibody incubation, which is substituted by PBS.

In another embodiment, the immobilization of anti-TNF-α (clone B-C7, Abcam®) at the surface of electrospun nanofibers was conducted by using EDC/NHS as a coupling agent. Prior to this step, the nanofibrous substrate was activated and chemically functionalized in order to make amine groups available to their bonding with the carboxyl groups present in the antibody. To determine the maximum immobilization capacity of the nanofibrous substrate, a wide range of primary antibody concentrations (concentrations of 0, 1, 2, 4, 6, 8 and 12 μg/ml) was tested. To determine the spatial distribution of the antibody at the nanofiber surface, the samples were observed at the fluorescence microscope.

### Mixed Immobilization of Two Antibodies

In an embodiment, the VEGF and bFGF antibodies are mixed in the same PBS solution at the concentrations optimized before, for a final volume of 200 μl per mesh. The antibodies mixture is incubated overnight at 4° C., and then the samples are washed twice with 0.1 M PBS (5 minutes each) and a 3% BSA incubation step for 30 min at RT is performed to block all the non-specific sites. The BSA solution is removed and the secondary antibody Alexa Fluor® 594 (for anti-VEGF) is incubated for 1 h at RT. The exceeding secondary antibody solution is collect for further quantification (n=3 samples, read in triplicate), as previously described, and the sample washed twice. The same approach is carried out for the secondary antibody Alexa Fluor® 488 (for anti-bFGF). Both secondary antibodies are prepared in a 1:200 concentration, diluted in PBS. A negative control sample is performed, without the immobilization of the primary antibodies, although all the other steps are done. All samples are analyzed under laser scanning confocal microscopy.

Side-by-Side Immobilization of Two Antibodies

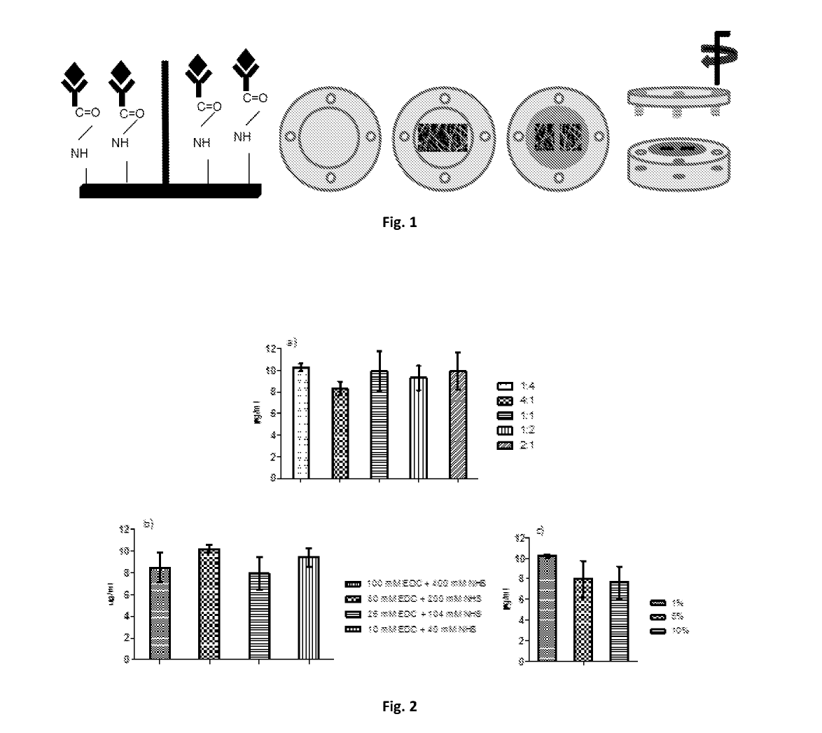

In an embodiment, in order to obtain a substrate with two different antibodies immobilized in a side-by-side design, a compartmental watertight chamber, represented in FIG. 1, is developed capable of physically divide a single 1 cm×2 cm functionalized electrospun NFM into two distinct areas, without allowing the mixture of the different antibodies solutions. Two different antibody solutions containing i.e. anti-VEGF and anti-bFGF are prepared at the concentrations described above and dropped over each area of the functionalized electrospun NFM. All the antibody immobilization steps, namely washings, BSA blocking and secondary antibody incubation, are performed, as previously described for the single antibody immobilization. The quantification of unbound secondary antibody is also performed and the samples recovered to characterize the spatial distribution of the antibodies by laser scanning confocal microscope.

### Laser Scanning Confocal Microscopy

Laser Scanning Confocal Microscopy can be conducted in order to characterize the spatial distribution of the antibodies at the surface of the electrospun NFMs. The fluorescent labeled biological molecules are analyzed by selecting the appropriate wavelengths: excitation at 495 nm for Alexa Fluor® 488 and 590 nm for Alexa Fluor® 594, and emissions at 570 nm for the red channel and 540 nm for the green channel. The single and multiple, either mixed or side-by-side, antibodies immobilized, i.e. TGF-β1, bFGF and VEGF, samples are placed onto glass slides and analyzed by laser scanning confocal microcopy (FluoView 1000, Olympus, Germany) at a 10× magnification.

### Recombinant and PL-Derived Growth Factor Quantification

Platelets Lysates: Preparation and Activation

In an embodiment, Platelet Rich Plasma (PRP) is provided by the Portuguese Blood Institute, which certifies the biological product accordingly to the Portuguese law. The number of platelets is counted and the sample volume is adjusted to 1 million platelets per μL. The collected PRP samples are then subjected to a 3 repeating temperature-shock cycles, i.e. frozen with liquid nitrogen at 196° C. and further heated at 37° C., and the remaining platelets are eliminated by centrifugation. A pool of Platelet Lysates (PL) is stored at −20° C. until further use. At the time of each experiment, a PL solution is thawed and filtered with a 0.22 μm filter.

Fluorescence-Linked Immunosorbent Assay (FLISA)

In an embodiment, after completing all the antibody immobilization steps previously described, 200 μl of the recombinant human protein solutions at different concentrations, ranging from 0 μg/mL to the concentration of each primary antibody, are incubated for 1 h at RT. The unbound recombinant human protein solutions are collected and stored at −20° C. until further quantification by ELISA. Two 0.1M PBS washing steps, of 5 minutes each, are performed and the biofunctionalized Nanofibrous substrates are incubated overnight at 4° C. with the corresponding primary antibody. After removal of the exceeding primary antibody solutions, the biofunctionalized substrates are washed again with PBS, another BSA blocking step is performed and the corresponding secondary antibody is incubated for 1 hour at RT. The fluorescence of unbound secondary solutions is also read out in a microplate reader (Synergy HT-BioTek). When the PL is used as the natural source of GFs, the same procedure is followed, although the recombinant human protein solution is substituted by 200 μl of PL.

Enzyme-Linked Immunosorbent Assay (ELISA)

In an embodiment, for the unbound GFs quantification, human basic-FGF and VEGF development ELISA kits from PrepoTech (Rochy Hill, N.J.; USA) are used, whereas the human TGF-β1 ELISA kit is from Boster Biological Technology (Fremont, Ca; USA). For the bFGF and VEGF development ELISA kits, the primary antibodies are firstly incubated overnight in a 96-well plate (Nunc-Immuno™MicroWell™ 96 well solid plates, Sigma Aldrich). All solutions are prepared according to the manufacturer protocol and, in the last step, 100 μL of an 2,2′-azino-bis(3-ethylbenzthiazoline-6-sulphonic acid (ABTS) liquid substrate is added to each sample and each plate read at 405 nm and 650 nm, respectively. The TGF-β1 ELISA is a ready-to-use kit, where the bottom wells are previously coated with the antibody. Both the standards and the samples are incubated and the assay conducted according to the protocol of the manufacturer protocol. In the last step of the procedure, 100 μl of the 3,3′,5,5′-Tetramethylbenzidine (TMB) stop solution is added to each well and the absorbance at 450 nm is read out (Synergy HT, Bio-TEK).

### Recombinant Inflammatory Molecule Quantification

At the end of the steps needed to primary antibody immobilization 200 μl of recombinant human TNF-α protein (Life Technologies) solution at concentration of 6 μg/ml was incubated for 1 h at room temperature. The supernatant of this recombinant protein was then collected and stored at −20° C. until further quantification by ELISA.

### Biological Assays

Cell Culture and Seeding

In an embodiment, a human pulmonary microvascular endothelial cell line (HPMEC-ST1.6R) is used to validate the developed biofunctionalized nanofibrous substrate. This cell line is used to study in vitro angiogenic process.

HPMEC-ST1.6R cells are cultured with M199 medium (Sigma Aldrich) supplemented with 20% FBS (Alfagene), 2 mM Glutamax (Life Technologies), Pen/Strep (100 U/100 g/mL; Life Technologies), heparin (50 μg/mL; Sigma Aldrich), Endothelial cell growth supplement (ECGS—25 μg/mL; Becton Dickinson) and incubated at 37° C. in a humidified 5% CO₂ atmosphere. HPMEC-ST1.6R cells are used at passages 30-32. Medium is changed twice a week until cell reaches 90% of confluence. Then, cells are harvested and seeded onto the activated and functionalized electrospun NFMs.

The electrospun PCL NFMs is sterilized by ethylene oxide at Pronefro® Produtos Nefrologicos, S.A. (Porto, Portugal). For NFM_AB1, NFM_Ab2, NFM-VEGF_{Rec} and NFM+VEGF_{PL}, VEGF antibody is immobilized overnight and, after the BSA blocking step, human recombinant protein (VEGF=4 μg/mL) and PL is incubated. Cell seeding is performed by dropping a 50 μl cell suspension containing 50 000 cells per substrate and left overnight. After cell attachment, culture medium is added to each type of cells and conditions. Untreated electrospun PCL NFMs (NFM) and NFMs where nanofibers are subjected to surface activation, aminolysis and primary antibody immobilization (NFM_Ab1) are used as controls. After 1, 3 and 7 days of culture, samples are collected for cell viability assay, DNA and total protein quantification and VEGF quantification.

Cell Viability

In an embodiment, the metabolic activity of HPMEC-ST1.6R cells seeded on untreated electrospun PCL NFM, NFMs with primary antibody immobilization, and biofunctionalized nanofibrous substrates (recombinant and PL-derived) is determined by the MTS assay (CellTiter 96 ® AQᵤₑₒᵤₛ One Solution, Promega). At days 1, 3 and 7, the culture medium is removed and the samples are rinsed with sterile PBS. A mixture of culture medium and MTS reagent (5:1 ratio) is added to each mesh, as well as to the negative control comprising no cells or samples. All conditions are performed in triplicate and left to incubate for 3 h, at 37° C. in a humidified 5% CO₂ atmosphere. Thereafter, the absorbance of the MTS reaction medium from each sample is read in triplicate at 490 nm (Synergy HT, Bio-TEK).

Cell Proliferation

In an embodiment, cell proliferation was determined by using a fluorimetric dsDNA quantification kit (Quant-iT′, PicoGreen®, Molecular Probes, Invitrogen, USA). The samples were collected at days 1, 3 and 7, washed twice with sterile PBS and transferred into eppendorf tubes containing 1 mL of ultrapure water. These samples were frozen at −80° C. until further analysis. Prior to DNA quantification, the various specimens for each samples were thawed and sonicated for 15 min. DNA standards were prepared at concentrations ranging from 0 to 2 μg/mL. Per each well of an opaque 96-wells plate (Falcon) were added 28.7 μL of sample or standard (n=3), 71.3 μL of PicoGreen solution and 100 μL of TE buffer. The plate was incubated for 10 min in the dark and the fluorescence was measured in a microplate reader (Synergie HT, Bio-Tek; USA) by using an excitation wavelength of 480 nm and an emission wavelength of 528 nm. The DNA concentration of each sample was calculated using a standard curve relating DNA concentration and fluorescence intensity.

Total Protein Synthesis

In an embodiment, samples were collected and prepared for assaying, as previously described in the Cell Proliferation. For the quantification of total protein synthesis, a Micro BCA™ Protein Assay Kit (Pierce, Thermo Scientific) was used. The assay was made accordingly to the manufacturer instructions. Briefly, standards were prepared at various concentrations ranging from 0 μg/mL to 40 μg/mL in ultra pure water. 150 μL of both samples and standards were assayed in triplicate and 150 μl of working reagent were further added to each 96-well plate. The plate was sealed and incubated for 2 hours at 37° C. The plate was left to cool down to RT and, thereafter, the absorbance at 562 nm was measured in a microplate reader (Synergy HT, Bio-Tek).

Statistical Analysis

In an embodiment, statistical analysis are performed using Graph Pad Prism Software.

Differences between the different conditions of the cellular assays are analyzed using non-parametric test (Kruskal-Wallis test) and a p<0.05 is considered significant. Data are presented as mean±standard deviations.

The main goal of these tests is to activate and functionalize the surface of electrospun PCL nanofibrous meshes to allow the binding of specific growth factors from a pool of different proteins. The biological fluid used in the tests is platelet lysate (PL). To achieve that selective binding, specific antibodies are immobilized at the nanofibrous substrate surface assuring that only the growth factors of interest are immobilized. The covalent immobilization is the preferred methodology to immobilize the antibodies at the surface of the chemically modified electrospun PCL nanofiber meshes (NFMs). Herein carboxyl groups and amines are used as they tend to react leading to an efficient covalent immobilization. Particularly, there is a carboxyl group (—COOH) at the end of the non-variable region of the antibodies that react with the amine groups (—NH₂) that are previously inserted at the surface of electrospun nanofibers, leading to the covalent immobilization of an antibody to the polymeric substrate. In this immobilization procedure some steps are optimized such as the coupling agent EDC/NHS ratio and concentrations, the maximum immobilization capacity of the activated and functionalized electrospun nanofibers, and the GFs binding capacity of the nanofibrous substrate. An endothelial cell line is used as a living model to assess the bioactivity of bound VEGF. The successful single antibody immobilization strategy is then transposed to different spatial configurations, by the immobilization of two antibodies, i.e. anti-bFGF and anti-VEGF, in the same nanofibrous substrate, in a mixed or in a side-by-side fashion.

From the most reported biomolecules present in PL, i.e. TGF-β1, PDGF-ββ, bFGF, EGF, IGF, and VEGF, TGF-β1, bFGF and VEGF are selected to conduct the validation experiments. TGF-β1 has an important role in promoting the production of ECM and in enhancing the proliferation of both fibroblasts and osteoblasts, being therefore relevant for both bone and cartilage strategies. bFGF is a potent inductor of cell proliferation, promoting angiogenesis and differentiation, as well as collagen production. It has a significant function in bone, cartilage and periodontal tissues. VEGF is a promoter of angiogenesis and proliferation of endothelial cells, playing a pivotal role in vascularization and stem cell differentiation.

### Optimization of Antibodies Immobilization

EDC/NHS Ratio and Concentrations

In an embodiment, a defined antibody is immobilized at the surface of electrospun nanofibers by a covalent bound mediated by a coupling agent, in this particular case the EDC/NHS mixture. Prior to the immobilization step, the electrospun PCL NFMs needs to be chemically modified by the insertion of amine groups that can react specifically with the carboxyl groups of the antibody. It is known that EDC alone is able to increase the immobilization efficiency of biomolecules. However, with the addition of NHS, a two-step reaction occurs and the presence of NHS forms semi-stable amines, enhancing the immobilization efficiency of the antibodies at the surface of a substrate. In order to ensure that the nanofibrous substrate is being used at its maximum immobilization capacity, different parameters concerning these two coupling reagents are tested.

The first parameter assessed is the ratio between the EDC/NHS coupling agents. Different ratios are tested, as represented in FIG. 2 a) and the 1:4 ratio presents the highest immobilization efficiency and lower variability being therefore selected for further experiments. Looking at the different ratios it seems that higher concentrations of NHS lead to a higher efficiency of immobilization. With the selected ratio, the next step is to optimize the concentration of each individual coupling agent, as represented in FIG. 2 b). From the four concentrations tested, the one that yields less variability and higher efficiency is the 50 mM EDC+200 mM NHS and, therefore, is the one selected for further experiments. The final step of this optimization process relies on testing the concentration of the EDC/NHS mixture in the final antibody solution. The EDC/NHS concentration that gets higher immobilization efficiency is the 1% (v/v), since the amount of immobilized antibody decreases with the increase of EDC/NHS concentration, as represented in FIG. 2 c). Based on these results, all further experiments concerning antibodies immobilization use the following optimized conditions: 1:4 ratio, 50 mM EDC+200 mM NHS concentrations and 1% (v/v) EDC/NHS concentration in the primary antibody solution.

### Single Antibody Immobilization at the Nanofibrous Surface

Antibodies Immobilization Efficiency

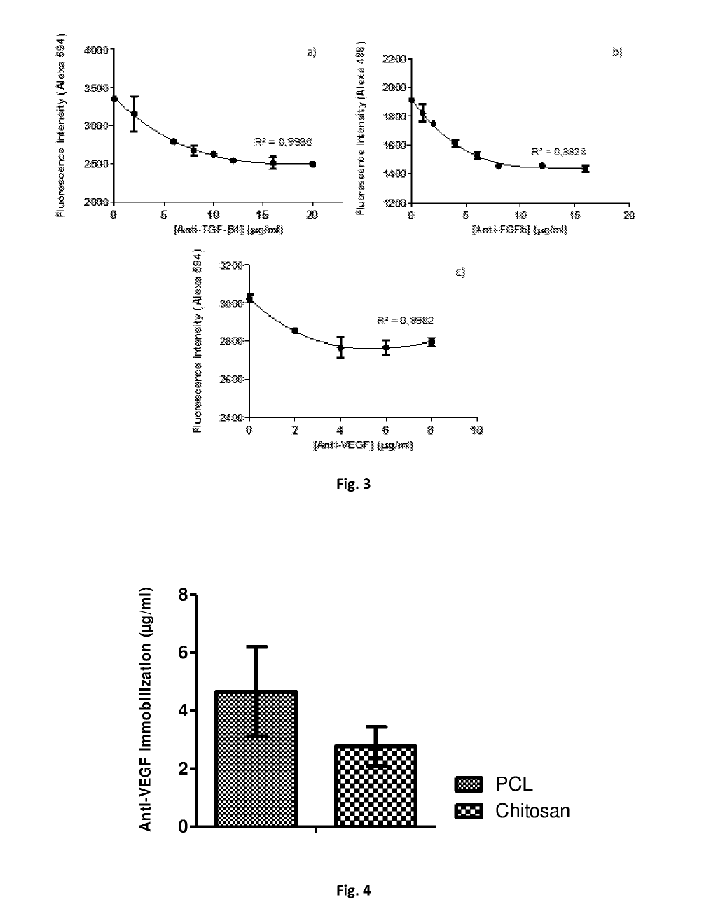

In an embodiment, the antibodies against the growth factors TGF-β1, bFGF and VEGF are immobilized at the surface of activated and functionalized electrospun nanofibers, in a wide range of concentrations, i.e. 0-20 μg/mL, to determine the maximum immobilization capacity of the nanofibrous substrate for each antibody. To achieve that purpose, an indirect quantification method is used, based on the measurement of unbound secondary antibody fluorescence, after its incubation with the immobilized primary antibody. As observed in FIG. 3, the higher amount of immobilized primary antibody corresponds to the lower fluorescence signal of the free secondary antibody. When the fluorescence signal reaches a plateau, the nanofibrous substrate presents the maximum concentration of immobilized primary antibody, reaching the saturation of the system. With the anti-TGF-β1, the maximum concentration of immobilized primary antibody is 12 μg/mL; in the case of anti-bFGF, the maximum capacity of the nanofibrous substrate is 8 μg/mL; whereas with the anti-VEGF its concentration reaches a value of 4 μg/mL. As clearly noticed, the different antibodies have different densities over the same activated and functionalized nanofibrous substrate. This observation may be related with the different sizes of the primary antibodies used. According to manufacturer's data the size of anti-VEGF is approximately 22 kDa and anti-bFGF is 17 kDA. In the case of the anti-TGF-β1 no information is given by the company although according to other manufacturers, the molecular weight of anti-TGF-β1 should be around 12-14 kDA. The immobilization data correlates with the antibodies size, since the more surface area is occupied by the antibody, the lower is its concentration at the nanofibers surface. Therefore, further experiments are performed with the concentrations of the primary antibodies that lead to the maximum immobilization capacity of the nanofibrous substrate. Antibodies are immobilized in different subtracts as referred before in a similar fashion as the common ELISA methods, that also involve the immobilization of specific antibodies at the bottom of the well to quantify the amount of specific antigen existing in a sample. From the datasheet of the ELISAs, referred above, it is possible to conclude that the values for anti-bFGF and anti-VEGF immobilization is 1 μg/mL and 0.5 μg/mL, respectively. The ELISA tests are conducted in 96 well-plates having approximately 0.32 cm² of surface area per well. In the case of the developed biofunctionalized nanofibrous substrate the apparent surface area is about three times higher (1 cm²) and despite having the same ratio between them the immobilization capacity is 8 times (8 μg/mL for anti-bFGF and 4 μg/mL for anti-VEGF) higher than in the standard ELISAs.

From FIG. 4 it is possible to conclude that both polymers (chitosan and PCL) were able to immobilize the desired antibodies (in this case anti-VEGF).

Primary Antibodies Standard Curve

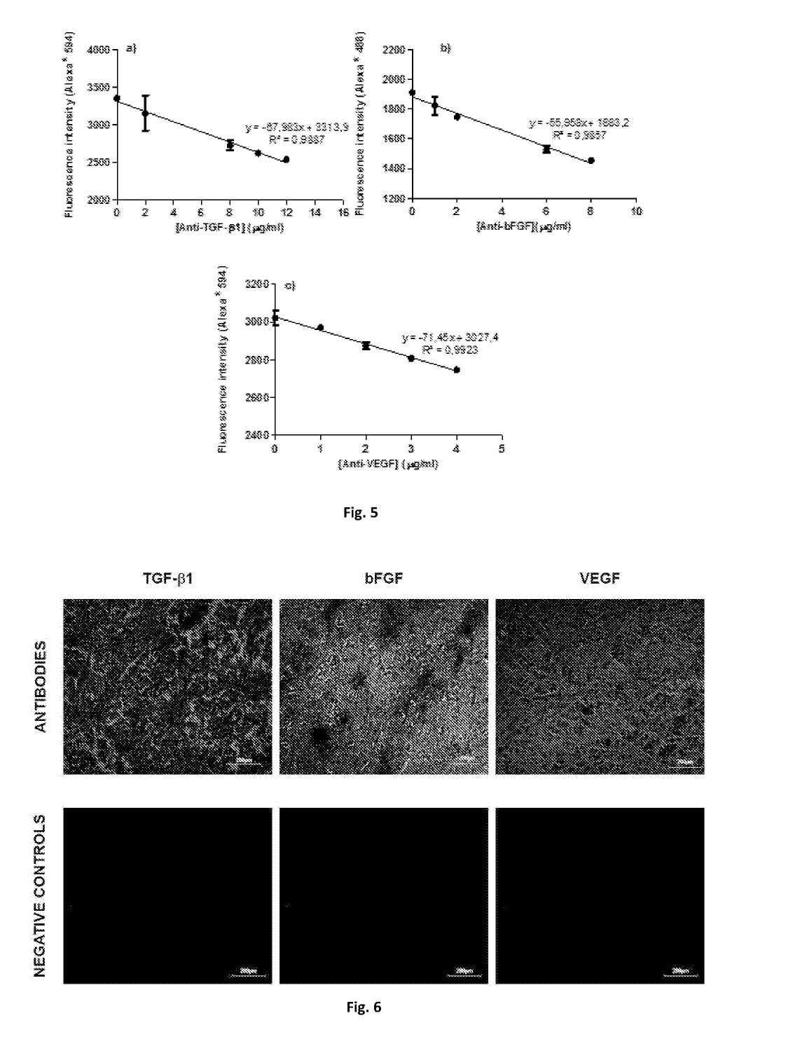

After determining the maximum antibody concentration immobilized at the surface of the activated and functionalized electrospun nanofibers, a standard curve can be determined for each antibody. With the remaining solution of each secondary antibody, it is possible to determine the amount of unbound secondary antibody, leading indirectly to the concentration of primary antibody that is immobilized. A linear regression standard curve fitting those data points allows obtaining a R² above 0.98 for every antibody, as represented in FIG. 5. These standard curves are further used for the quantification of the immobilized antibodies in next assays.

Spatial Distribution of Antibodies at the Surface of Electrospun Nanofibers

FIG. 6 shows the spatial distribution of the TGF-β1, bFGF and VEGF antibodies at the surface of activated and functionalized electrospun nanofibers. Primary antibodies are immobilized at the previous optimized concentrations: in FIG. 6 a) 12 μg/mL of anti-TGF-β1, in FIG. 6 b) 8 μg/mL of anti-bFGF and in FIG. 6 c) 4 μg/mL of anti-VEGF. In the case of the TGF-β1 and VEGF antibodies, the secondary antibody Alexa Fluor® 594 is used, whereas the secondary antibody Alexa Fluor® 488 is used for the bFGF antibody. The negative controls represented in FIG. 6 d), e) and f) are subjected to all the steps except the incubation with the primary antibodies.

In an embodiment, all the immobilized antibodies seem to be uniformly distributed through the nanofibers surface, resembling the random mesh-like arrangement of the electrospun NFM structure. The TGF-β1 antibody seems to have a more intense and densely distributed fluorescence than the other immobilized antibodies, probably due to its higher concentration (12 μg/mL). To ensure that the secondary antibody only binds to the immobilized primary antibody, a control experiment is defined in which all the steps are performed except the incubation with the primary antibody. These conditions are analyzed for fluorescence, as represented in FIG. 6 d), e) and f) and no fluorescence is detected. Since no fluorescent signal is observed, it means that Alexa Fluor® 594 and Alexa Fluor® 488 secondary antibodies are not immobilized at the surface of activated and functionalized electrospun nanofibers, confirming the specific binding between these two secondary antibodies and the corresponding immobilized primary antibodies.

### Growth Factors Binding Capacity to the Biofunctionalized Nanofibrous Substrate

Quantification of Bound Recombinant Proteins

In an embodiment, after confirming the specific immobilization of the TGF-β1, bFGF and VEGF antibodies and determining the corresponding standard curves, it is assessed the binding capacity of the biofunctionalized nanofibrous substrates. Namely it is characterized the total amount of each growth factor that a functionalized mesh can bind. For that, two different growth factor (GF) sources are tested: (i) recombinant proteins to evaluate the maximum binding capacity of the biofunctionalized nanofibrous substrate, and (ii) PL-derived GFs to assess the selective binding capacity of the biofunctionalized nanofibrous substrate. In fact, when using recombinant protein, it is known that the only protein competing to the primary antibody is the one being tested. In PLs there is a complex mixture of proteins competing for the antibodies, thus demonstrating the specificity of the bound proteins.

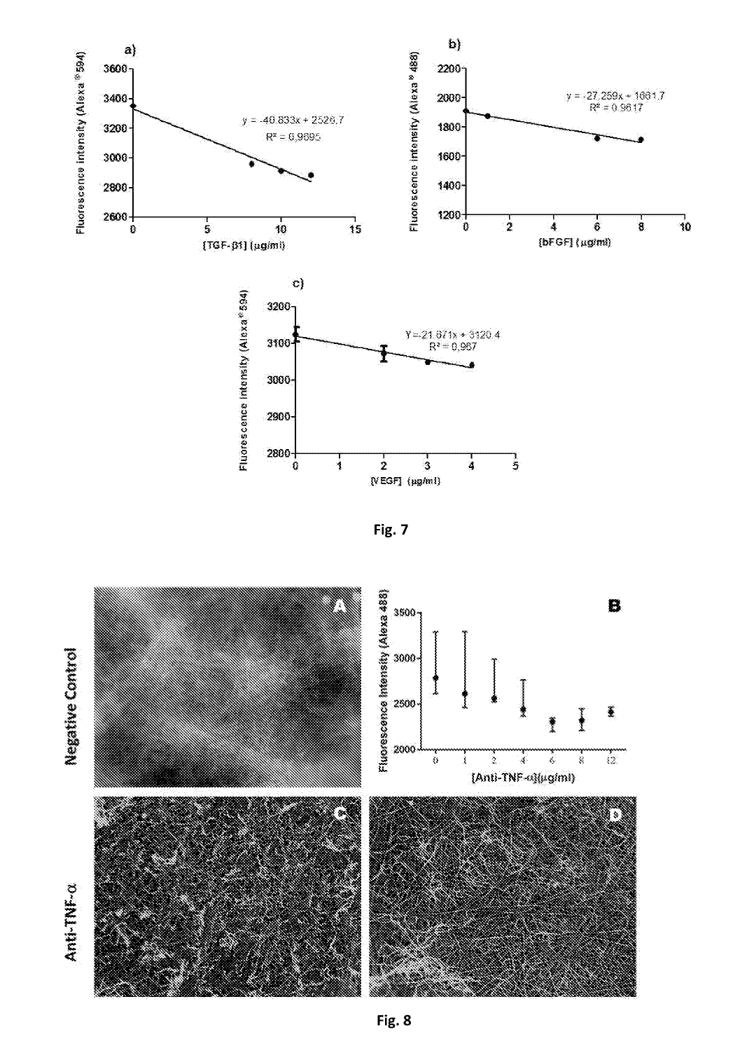

Following the immobilization of each antibody at the surface of the activated and functionalized electrospun nanofibers, the corresponding recombinant protein is added at different concentrations, varying from 0 μg/mL to values higher than the concentration of the previously immobilized primary antibody. However, for all the three antibodies in study, the biofunctionalized nanofibrous substrate starts to reach its maximum GF binding capacity near to the higher concentration of the primary antibody as showed in FIG. 7, i.e. 12 μg/mL for TGF-μ1, 8 μg/mL for bFGF and 4 μg/mL for VEGF. The results are also confirmed and quantified with commercial available ELISA and the maximum loading capacity corresponded to the above mentioned values, reaching around 100% of loading efficiency for all the three GFs. As observed in FIG. 7, an increase in the amount of recombinant protein leads to a decrease in the fluorescence signal, meaning that less secondary antibodies are unbound. It is important to notice that each GF-bound to the biofunctionalized nanofibrous substrate has its own slope or rate. The higher slope is observed for TGF-β1 (40,833), followed by the one for bFGF (27,259) and the lower one for VEGF (21,771). That may rely on the fact that higher concentrations of primary antibodies are occupied faster by the corresponding recombinant protein, leading to a rapid decrease on the fluorescence signal of the unbound secondary antibody. TGF-β1 has been previously reported to be immobilized in different substrates like gelatin and magnetic beads, whereas bFGF has been immobilized into different platforms like, PEG hydrogel, PLGA films, and PLLA+collagen scaffolds. VEGF has been covalently immobilized into different substrates such as collagen scaffolds, PLGA and hydrogels. From all this data, it was possible to conclude that the nanofibrous substrate of the present invention enables immobilizing a higher concentration of GFs at the order of g/mL, whereas most of the other systems report values that are at the magnitude of ng/mL, reflecting the positive effect of the increased surface area of the electrospun nanofibers over the bound GFs.

Quantification of Bound PL-Derived Growth Factors

In an embodiment, the amount of each GF of interest in the PL by an ELISA kit is quantified. Table 1 shows the range of concentrations obtained from two different human samples. Comparing to other values reported in the literature, TGF-β1 (169.9±84.5 ng/mL) is about 15 times higher than the ones obtained with the samples described in the present invention, whereas the values of VEGF are comprised in the values 0.076 to 0.854 μg/mL as reported in Alsousou, J., Thompson, M., Hulley, P., Noble, a & Willett, K. The biology of platelet-rich plasma and its application in trauma and orthopaedic surgery: a review of the literature. J. Bone Joint Surg. Br. 91, 987-96 (2009). Despite being described as one of the most abundant GFs of PRP samples, no data reporting the concentration of bFGF was found in the literature. The differences of the quantified GFs and its variability are related with the differences between donors leading to different concentrations of the GFs of interest.

TABLE 1
Quantification of the growth factors of interest derived
from two human PL samples

| Growth | Concentration in PL samples | | % Binding | |
|---|---|---|---|---|
| Factors | Donor 1 | Donor 2 | Donor 1 | Donor 2 |
| TGF-β 1 | 4.2 ng/mL | 11.05 ng/mL | 83.92 ± 2.68 | 86.85 ± 3.26 |
| bFGF | 8.6 ng/mL | 102.5 ng/mL | 54.78 ± 4.75 | 63.97 ± 3.48 |
| VEGF | 0.0949 ng/mL | 0.4263 ng/mL | 49.52 ± 3.05 | 58.85 ± 4.02 |

After determining the recombinant human GF binding capacity of the activated and functionalized nanofibrous substrate, it is tested for the selective binding of GFs derived from the PL samples. ELISAs are performed to determine the amount of bound autologous GFs for two different donors. For PL-derived TGF-β1, the binding efficiency, around between 84%-87%, is not as high as in the case of the recombinant protein. Considering the bFGF, only around 55%-64% of PL-derived protein is bound to the nanofibrous substrate immobilized primary antibody. The same trend is observed for the VEGF, where about 50%-58% of PL-derived VEFG is bound by the corresponding immobilized antibody. Despite the concentrations of the GFS in the different samples the bounding of the GFs stay in the same range for the two donor samples, showing the consistency of the method.

Despite the order of magnitude differences in the concentration of GFs present in the PL, ranging from pg/mL for VEGF to ng/mL for TGF-β1 and bFGF, those concentrations are much lower than the ones determined for the maximum binding capacity of the biofunctionalized nanofibrous substrate where recombinant proteins are used at μg/mL. The inability of the biofunctionalized nanofibrous substrate to immobilize 100% of the GFs amount present in the PL can be related with the fact that this biological fluid is highly rich in different GFs and proteins that can compete to the binding sites of each immobilized antibody. Another technical aspect that can justify the binding of GFs derived from the PL in the range of 50-87% is the detection limits of the used ELISAs, which do not enable detecting the GFs at very low concentrations (16 pg/mL for VEGF ELISA Kits, 63 pg/mL for bFGF and 15.6 pg/mL for TGF-β1).

### Immobilization and Binding Capability of Anti-TNF-α Functionalized Nanofibrous Substrate

In an embodiment, once the performed assay represents the freely available secondary antibody fluorescence on the supernatant, it's reasonable to assume that a higher amount of immobilized primary antibody corresponds to a lower fluorescence signal of the unbound secondary antibody. Therefore, the maximum immobilization of the anti-TNF-α was reached when the fluorescence intensity values reach a plateau, as the concentration of primary antibody increases. Thus, analyzing the FIG. 8 B, the maximum concentration of immobilized primary antibody lays in 6 μg/ml once the fluorescence intensity is higher both on right and left side of this value. Furthermore, by comparing the negative control (FIG. 8 A) with the nanofibrous substrate with immobilized antibody at a concentration of 6 μg/ml (FIG. 8 C and FIG. 8 D), it's possible to observe the distribution of anti TNF-α along the electrospun nanofibers, in contrast to the non-specific fluorescence observed in the negative control.

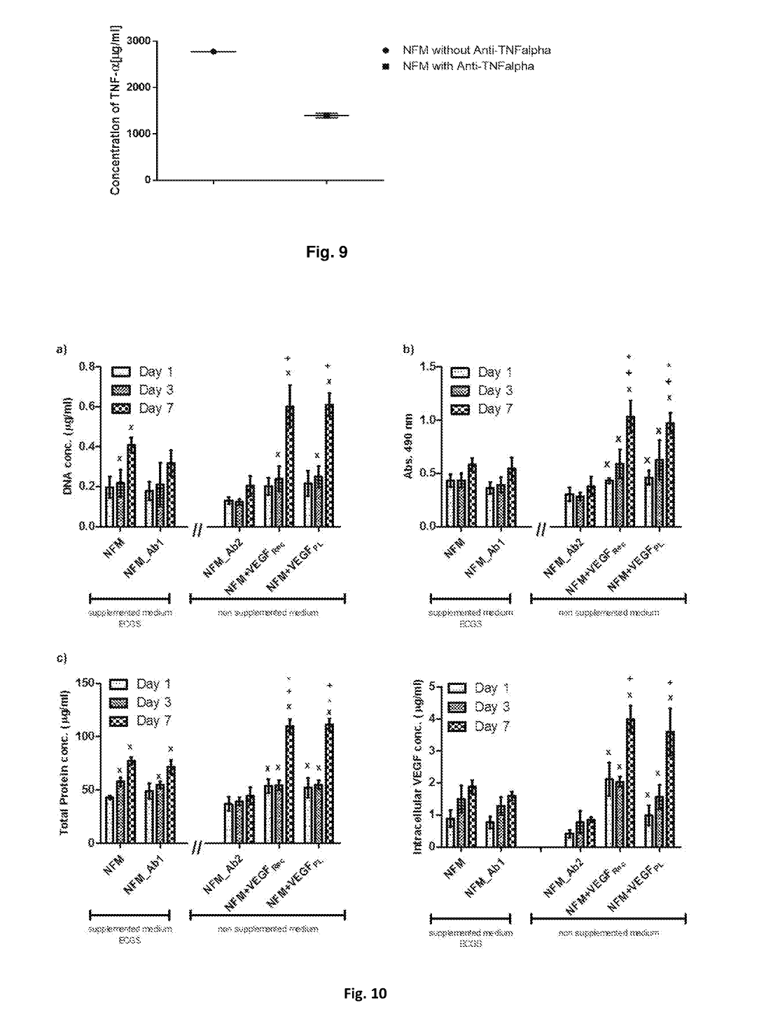

In an embodiment, to determine the capacity of the nanofibrous substrate to bound soluble recombinant TNF-α, two sets of test conditions were prepared: a group of nanofibrous substrate without immobilized anti-TNF-α (negative control), and another with anti-TNF-α immobilized at the optimal concentration previously determined (i.e. 6 μg/ml). The FIG. 9 shows the amount of unbound TNF-alpha present in the supernatant, after one hour incubation.

By observing the graphic of FIG. 9 it's possible to determine that, comparing to the negative control (0 ug/ml of anti-TNF-α), the functionalized nanofibrous substrate with immobilized anti-TNF-α (at a concentration of 6 μg/ml), provides a decrease of approximately 50% of the soluble recombinant TNF-α.

### VEGF Biological Activity

In an embodiment, to confirm that the covalent immobilization method do not compromises the bioavailability of the antigen binding site of the antibodies and the behavior of the bound growth factors the bioactivity of bound VEGF is assessed. VEGF has been described to induce vascularization and angiogenesis so human pulmonary microvascular endothelial cells (HPMEC-ST1.6R cell line) are seeded onto the biofunctionalized electrospun nanofibrous substrates. VEGF condition are selected since VEGF is less concentrated factor in Platelet Lysate so the bioactivity for the worst (less concentrated) scenario is assessed. Different biological assays are conducted to assess the endothelial cells viability and proliferation, the total protein synthesis, as well as the quantification of intracellular synthesis of VEGF, as represented in FIG. 10. The endothelial cells are cultured at the surface of 5 different substrate conditions: i) untreated electrospun PCL NFM (NFM), ii) NFM with primary antibody (NFM_Ab1), iii) electrospun NFM with bound recombinant VEGF (NFM+VEGFRec), iv) electrospun NFM with PL-derived VEGF (NFM+VEGFPL) and v) NFM with primary anti-VEGF (NFM_Ab2). In conditions i) and ii) the medium is supplemented with Endothelial Cell Growth Supplement (ECGS), a mixture of growth factors aimed to stimulate the growth of human and animal vascular endothelial cells. In conditions iii), iv) and v) the endothelial cells are cultured in basal medium since the aim is to evaluate the actions of the immobilized form of VEGF so the medium is not supplemented by other GFs. Statistical analysis is performed for the five different conditions comparing each time point, i.e. Day 1, Day 3 and day 7. Data is considered statistical different for p values<0.05. (*) denote significant differences when compared to NFM condition, (⁺) when compared to NFM_Ab1 supplemented media, (^{X}) when compared to NFM_Ab2, (^{#}) when compared to NFM_VEGF and (^{&}) when compared to NFM_PL.

The data confirms the biological activity of the bound VEGF, since in all the assays performed (cell proliferation, cell viability, total protein synthesis and intracellular VEGF), significant differences between the NFM+VEGF_{Rec} and NFM+VEGF_{PL} are always reported when compared to NFM_Ab2 this condition only differs from the previous one by not having an immobilized protein. This observation demonstrates that bound VEGF, recombinant or PL-derived, indeed make a difference in the biofunctionalized nanofibrous substrate. Furthermore, no significant differences are observed at first day, regarding the cell DNA content, as shown in FIG. 10 a), confirming the similar number of cells seeded over the different substrate. Since there are no significant differences between NFM+VEGF_{PL} and NFM+VEGF_{Rec} even at a much higher concentration of recombinant VEGF (4 μg/mL) it is possible to conclude that the amount of protein derived from PL samples (0.251 μg/mL) is enough to induce cells proliferation. Although an excess of immobilized recombinant protein did not have adverse effects on the endothelial cells. The controls do not present significant differences among them showing that the chemical treatment does not affect cells behavior. Another important aspect of these cellular experiments is the observation of significant differences between the bound VEGF and the conditions where the GFs are given in its soluble form (NFM and NFM_Ab1) demonstrating its bioactivity along time. This same trend has already been reported showing some evidences that the immobilized form can be beneficial for endothelial cells metabolic activity and protein synthesis. This is probable due to the fact that immobilized VEGF can provide a more controlled and sustained influence over the cells, comparing with the transient effect of soluble VEGF. GFs immobilization in substrates also promotes a local regulation and control over cellular activity, as expressed by the intracellular VEGF over expression. Furthermore, immobilized growth factors can provide extended signaling since the ligant is internalized as a ligant and/or receptor complex. Covalent attachment of angiogenic growth factors to biomaterial scaffolds is an advantageous strategy for the development of polymeric matrix with enhanced angiogenic capabilities.

### Immobilization of Multiple Antibodies in Different Spatial Configurations

An embodiment comprises immobilizing more than one antibody at the surface of a single activated and functionalized NFM. Two different immobilization embodiments are presented below: one with the mixed distribution of defined antibodies, i.e. anti-VEGF and anti-bFGF, and another with side-by side localization of those distinct antibodies, in different areas of the same nanofibrous substrate. With the immobilization of multiple antibodies at the surface of the same nanofibrous substrate it is expected to develop a highly efficient system for designing advanced strategies for diverse cell biology, tissue engineering and regenerative medicine.

Mixed Immobilization of Two Different GFs

In an embodiment, the purpose of the mixed immobilization is to have, at the surface of the same nanofibrous substrate, two different but complementary antibodies, specifically the anti-bFGF and anti-VEGF. To implement this strategy, the antibodies concentrations optimized before (i.e. 8 g/mL for bFGF and 4 μg/mL for TGF-β1) are used and incubated simultaneously.

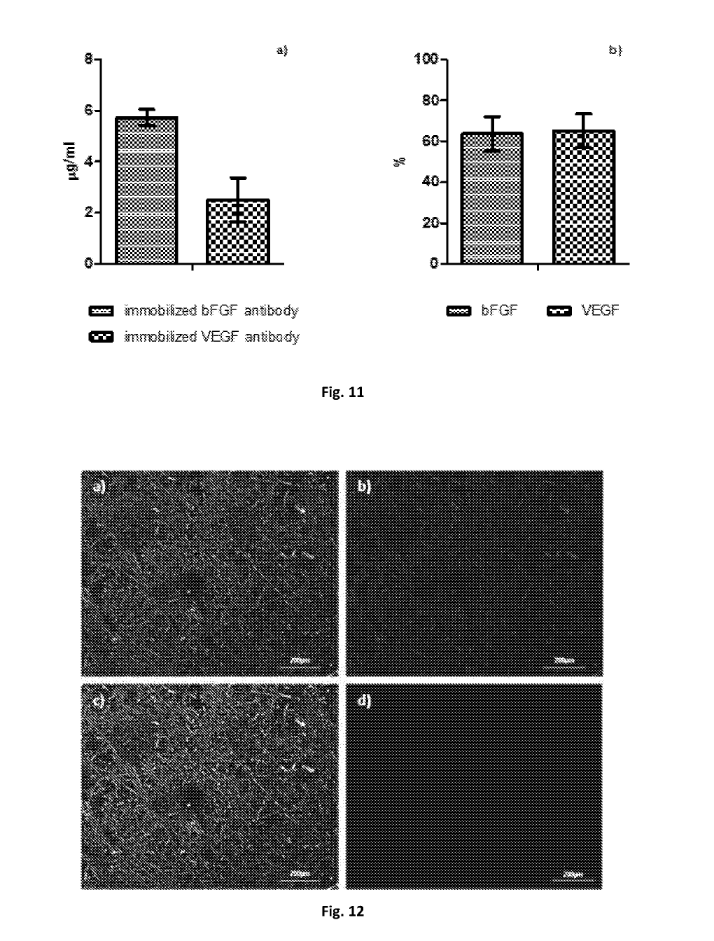

FIG. 11 a) shows how much of the initial antibodies concentrations have been immobilized, being these results obtained by applying the determined standard curves for the single antibody immobilization strategy, as shown in FIG. 5. In the case of anti-bFGF, around 63% of the initial antibody concentration is immobilized, whereas around 72% of the initial concentration is immobilized in the case of VEGF antibody. These immobilization efficiencies are above the expected outcomes, since the two antibodies, although at different concentrations, are competing for the same amount of NH₂ available at the surface of an activated and functionalized nanofibrous substrate. The antibodies can be immobilized until the system reaches its maximum capacity, not having more chemical groups available for the antibodies immobilization. Therefore, in the mixed distribution of two antibodies is reasonable that the immobilization efficiency could not be as high as for the single immobilization strategy. Human PL samples are also incubated with the mixed immobilization nanofibrous substrate to evaluate if this system is able to bind selectively and simultaneously the two growth factors of interest. As represented in FIG. 11 b), the binding efficiency is about 64% for bFGF and 65% for VEGF.

To evaluate the spatial distribution and confirm that both antibodies are indeed immobilized at the same nanofibrous substrate in a mixed design, the corresponding secondary antibodies are used and their fluorescence observed at the laser scanning confocal microscope.

FIG. 12 a) and b) represent the Alexa Fluor®488 and the Alexa Fluor® 594 fluorescent antibodies bound to the anti-bFGF and anti-VEGF at the surface of activated and functionalized nanofibrous substrate. The bFGF and VEGF antibodies are simultaneously immobilized in the same mesh, at the previously optimized concentrations. Alexa Fluor® 448 is used as the secondary antibody for the anti-bFGF); and the Alexa Fluor® 594 is used for the anti-VEGF.

It is possible to observe that the antibodies are uniformly distributed over the functionalized nanofibrous substrate. However, it is also possible to notice that the green fluorescence is slightly more intense than the one concerning anti-VEGF immobilization. This may be related with the higher concentration of the immobilized anti-bFGF, which can lead to higher intensity of Alexa Fluor® 488 antibody fluorescence. FIG. 12 c) represents the spatial distribution of the two primary antibodies, and shows a merged view of the two different channels and/or fluorescences, i.e. green and red, corresponding to the VEGF and bFGF immobilized antibodies distribution. The merging of the two pictures yields a significant amount of yellow spots which demonstrate the co-localization of both antibodies in a mixed fashion, over the same nanofibrous substrate. Finally FIG. 12 d), showing activated and functionalized nanofibrous substrates without primary antibodies immobilization, presents the negative control of the experience, where the incubation step with the primary antibodies solution is not conducted to make sure that the secondary antibodies are only bind to the corresponding primary antibodies.

### Side-by-Side Immobilization of Two Distinct Antibodies

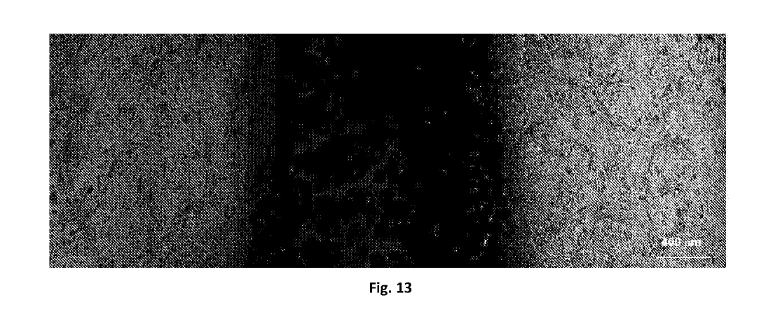

In an embodiment, with the side-by-side immobilization of two antibodies it is intended to demonstrate the possibility to have two distinct GFs selectively immobilized from biological fluids, bound side by side, having in mind their functional role over two distinct cell types spatially juxtaposed in physiological environments. In order to achieve this purpose, a compartmental watertight device is designed to enable creating two distinct areas in the same nanofibrous substrate. Each area of the activated and functionalized nanofibrous substrate is incubated with the defined primary antibody and, further with the corresponding secondary antibody, leading to the side-by-side configuration and distribution presented in FIG. 13. The reddish fluorescent area of the nanofibrous substrate corresponds to the anti-VEGF immobilization, whereas the green area reports to the bFGF antibody immobilization. The black area corresponds to the bar that separates the same nanofibrous substrate into the two areas. It is possible to detect local green spots or red dots which can be due to some diffusion of the antibodies solution trough the activated and functionalized nanofibrous substrate.

With this system it is possible to seed and culture two different cell types over the two areas of the biofunctionalized nanofibrous substrate, where defined antibodies and the corresponding GFs are previously immobilized. With this strategy it is possible to obtain tailored and advanced co-culture systems, allowing to study cell-cell interactions in vitro in the present of specific GFs.

The covalent immobilization method is successfully implemented in nanofibrous substrates, presenting different efficiencies depending on the antibody of interest. After the antibodies immobilization in different designs, the biofunctionalized nanofibrous substrates enables the binding of the corresponding growth factors, as well as to select a specific GF from a complex biological fluid, i.e. PLs, comprising a pool of different GFs and proteins. The bioactivity of the bound growth factors is confirmed by cell culture assays, and the beneficial outcomes of the nanofibrous substrate bound GFs are confirmed by biochemical data. The biological data suggests that this substrate offers unique possibilities to study basic cell biology as well as tissue engineering and regenerative medicine fields, since it is possible to specifically bind different GFs of interest at the surface of the nanofibrous substrate. Ultimately, using both biological fluids and cells from an autologous source, it will be possible to implement very effective and personalized therapies tailored for specific clinical conditions.

The present invention is not, in any way, restricted to the embodiments described herein and a person of ordinary skills in the area can provide many possibilities to modifications thereof without departing from the general idea of the invention, as defined in the claims. The following claims define further preferred embodiments of the present invention.

1. A polymeric substrate product for binding to antigens, wherein said product comprises a polymeric substrate having a specific surface area between 15-90 cm²/mg, said substrate comprises:
a functional group able to bind to an antibody, wherein the functional group is selected from a list consisting of: amine, sulfhydryl, carbonyl, carboxyl, or mixtures thereof, and
an antibody for binding to an antigen,
wherein the antibody is bound to the substrate through said functional group and the antibody concentration is more than 1 μg/mL.

2. The substrate according to claim 1, wherein the antibody is selected from a list consisting of: an antibody against a growth factor, an antibody against an inflammatory molecule, an antibody against a extracellular vesicle, an antibody against a hormone from a biological fluid, or mixtures thereof.

3. The substrate according to claim 1, wherein the antibody concentration is 1.5 μg/mL-20 μg/mL.

4. The substrate according to the claim 3, wherein the antibody concentration is 2 μg/mL-12 μg/mL.

5. The substrate according to the preceding claim wherein the specific surface area varies between 20-90 cm²/mg.

6. The substrate according to claim 1, wherein the polymeric substrate comprises a synthetic polymer selected from the list consisting of: polycaprolacton, polyhydroxyalkanoate, polyhydroxybutyrate, polyethylene adipate, polybutylene succinate, poly(3-hydroxybutyrate-co-3-hydroxyvalerate), polyethylene terephthalate, poly(ethylene), poly(styrene), poly(tetrafluoroethylene), poly(propylene), poly(pyrrole), poly(dimethyl siloxane), polydioxanone (PDA), poly(methyl methacrylate), and mixtures of the foregoing.

7. The substrate, according to claim 1, wherein the substrate comprises a natural polymer selected from the group consisting of: starch, chondroitin sulfate, hyaluronic acid, chitosan, alginate, dextran, agarose, cellulose, collagen, xanthan, carrageenan, aloe vera, gelatin, silk protein, fibronectin, and mixtures of the foregoing.

8. The substrate according to claim 1, wherein the polymeric substrate comprises a mixture of a synthetic and a natural polymer, wherein the synthetic and natural polymers comprise, respectively: polycaprolactone and starch, or polycaprolactone and hyaluronic acid, or polycaprolactone and chondroitin sulfate, or polycaprolactone and chitosan, or polycaprolactone and collagen, or polycaprolactone and gelatin, or polycaprolactone and silk protein, or polycaprolactone and fibronectin.

9. The substrate according to claim 1, wherein the covalent bound is mediated by a coupling agent.

10. The substrate according to claim 1, wherein the coupling agent is selected from the group consisting of: EDC, NHS, Sulfo-NHS, EDC-HCl, sulfo-SANPAH-Succinimidyl ester-phenyl azide, sulfo-LC-SPDP, cyanogen bromide sodium cyanoborohydride, carbonyl diimidazole, and mixtures of the foregoing.

11. The substrate according to claim 1, comprising at least one antibody against a growth factors selected from the group consisting of: TGF-α, TGF-β1, TGF-β2, TGF-β3, EGF, HIF, PDGF-AA, PDGF-AB, PDGF-BB, PDGF-CC, PDGF-DD, VEGF-A, VEGF-B, VEGF-C, VEGF-D, IGF-1, FGF-2, FGF-18, BMP-2, BMP-4, BMP-6, BMP-7/OP-1, CDMP-1/GDF-5, CDMP-2, and mixtures of the foregoing.

12. The substrate according to claim 1, comprising at least one antibody against an inflammatory molecule selected from the group consisting of: tumor necrosis factor-alpha, granulocyte-macrophage colony-stimulating factor, interferon-gamma, IL-1, IL-6, IL-7, IL-8, IL-10, IL-12, IL-15, IL-17A, IL-17F, IL-18, IL-19, IL-20, IL-21, IL-22 IL-23, IL-32, IL-33, and mixtures of the foregoing.

13. The substrate according to claim 1, comprising at least one antibody against an hormones selected from the group consisting of: prolactin, adrenocorticotropic hormone, angiotensinogen, thyroid-stimulating hormone, parathyroid hormone, Luteinizing hormone, follicle-stimulating hormone, melanocyte-stimulating hormone, thymosin, orexin, leptin, adiponectin, secretin, histamin, endothelin, gastrin, ghrelin, erythropoietin, oxytocin, vasopressin, proopiomelanocortin, triiodothyronine, thyroxine, calcitonin, parathormone, cholecystokinin, thyrotrophic hormone, growth hormone, corticotrophin, gonadotropin, melatonin, serotonin, epinephrine, norepinephrine, dopamine, dehydroepiand-rostendione, estrogen, progesterone, testosterone, dihydrotestosterone, estradiol, insulin, glucagon, cortisol, cortisone, corticosterone, aldosterone, inhibin and mixtures of the foregoing.

14. The substrate according to claim 1, comprising at least one antibody against a surface marker of an extracellular vesicular body selected from the group consisting of: CD2-associated protein, CD9 molecule, CD14 molecule, CD24 molecule, CD24a antigen, CD36 molecule, CD44 molecule, CD55 molecule, CD59 molecule, CD63 molecule, CD81 molecule, CD82 molecule, CD86 molecule, CD274 molecule, CD5 molecule-like, CD163 molecule-like 1, aquaporin 2, flotillin 1, Fas ligand, lysosomal-associated membrane protein 1, lysosomal-associated membrane protein 2, mannosyl-oligosaccharide glucosidase, flotillin 1, flotillin 2, intercellular adhesion molecule 1, tumor susceptibility gene 101, and transferrin receptor.

15. The substrate according to claim 1, wherein the antibody comprises a mixture of two or more antibodies against an autologous growth factor, or an anti-inflammatory agent, or a micro vesicular body, or an hormone present in a biological fluid: TGF-β1 and TNFalpha, or BMP-2 and estrogen, or FGF-2 and CD63, or IL-1 and cortisol, or TNFalpha and CD9, or estrogen and CD81, or TGF-β1 and TNFalpha and estrogen, or TNFalpha and estrogen and CD9, or TGF-β1 and IL-1 and CD63, or BMP-2 and estrogen and CD81.

16. The substrate according to claim 15, wherein the antibodies are spatially distributed in a random or aside-by-side fashion.

17. The substrate according to claim 1, wherein the polymeric substrate is fibrous.

18. (canceled)

19. (canceled)

20. A polymeric substrate composition for binding to antigens for use in the regeneration or treatment of tissues, wherein said composition comprises:
a polymeric substrate having a functional group able to bind to an antibody, wherein the functional group is selected from the group consisting of: amine, sulfhydryl, carbonyl, carboxyl, and mixtures of the foregoing, and
an antibody for binding to an antigen,
wherein the antibody is bound to the substrate through said functional group and the antibody concentration is more than 1 μg/mL.

21. A composition according to claim 20, wherein the antibody is selected from the group consisting of: an antibody against a growth factor, an antibody against an inflammatory molecule, an antibody against a extracellular vesicle, an antibody against a hormone from a biological fluid, and mixtures of the foregoing.

22. (canceled)

23. (canceled)

24. (canceled)

25. (canceled)

26. A skin patch comprising the polymeric substrate of claim 1.

27. A diagnostic kit comprising the polymeric substrate of claim 1.
